(19) United States
(12) Patent Application Publication
(10) Pub. No.: US 2025/0078997 A1
(43) Pub. Date: Mar. 6, 2025

(54) CLINICAL RECOMMENDATION METHOD, CLINICAL RECOMMENDATION APPARATUS, AND COMPUTER-READABLE RECORDING MEDIUM

(71) Applicant: AESOP Technology Inc., San Francisco, CA (US)

(72) Inventor: An Jim Long, Taipei City (TW)

(73) Assignee: AESOP Technology Inc., San Francisco, CA (US)

(21) Appl. No.: 18/029,639

(22) PCT Filed: Feb. 20, 2023
(86) PCT No.: PCT/US2023/013402

§ 371 Date: Mar. 30, 2023

(51) Int. Cl. G16H 50/20; G16H 10/60

(52) U.S. Cl. CPC G16H 50/20; G16H 10/60

(57) ABSTRACT

A clinical recommendation method, a clinical recommendation apparatus, and a computer-readable recording medium are provided. In the method, Each medical parameter is determined as an independent event. Each medical parameter has an actual diagnosis. The independent event is defined that a medical parameter is independent of the actual diagnosis thereof. Multiple reference probabilities of each medical parameter are determined based on a probabilistic model. Each reference probability is a probability of one medical parameter in a condition where one reference diagnosis occurs. Multiple final probabilities of reference diagnoses are determined according to the reference probabilities of the medical parameters. A recommendation is determined according to the final probabilities of the reference diagnoses. Accordingly, a proper recommendation may be provided.

### BACKGROUND

### Technical Field

The disclosure relates to a clinical evaluation technology, and in particular to a clinical recommendation method, a clinical recommendation apparatus, and a computer-readable recording medium.

### Description of Related Art

The multivariate model such as a neural network is a black box model. However, the black box model may be not suitable for clinical evaluation-related applications. There have higher privacy regulations for the medical data of patients. For example, the regulation is that the number of single data shall not be limited to less than or equal to 10 persons. However, it can not be proved by the black box model, so large medical data can not be implemented.

### SUMMARY

The embodiments of the disclosure provide a clinical recommendation method, a clinical recommendation apparatus, and a computer-readable recording medium, to be adapted for clinical evaluation-related applications.

In one of the exemplary embodiments, a clinical recommendation method includes (but is not limited to) the following. Each medical parameter is determined as an independent event. Each medical parameter has an actual diagnosis. The independent event is defined that a medical parameter is independent of the actual diagnosis thereof. Multiple reference probabilities of each medical parameter are determined based on a probabilistic model. Each reference probability is a probability of one medical parameter in a condition where one reference diagnosis occurs. Multiple final probabilities of reference diagnoses are determined according to the reference probabilities of the medical parameters. A recommendation is determined according to the final probabilities of the reference diagnoses.

In one of the exemplary embodiments, a clinical recommendation apparatus includes (but is not limited to) a memory and a processor. The memory is configured for storing a program code. The processor is coupled to the memory and is configured for executing the program code to perform the following. Determining each medical parameter as an independent event. Determining multiple reference probabilities of each medical parameter based on a probabilistic model. Determining multiple final probabilities of reference diagnoses according to the reference probabilities of the medical parameters. Determining a recommendation according to the final probabilities of the reference diagnoses. Each medical parameter has an actual diagnosis. The independent event is defined that a medical parameter is independent of the actual diagnosis thereof. Each reference probability is a probability of one medical parameter in a condition where one reference diagnosis occurs.

In one of the exemplary embodiments, a non-transitory computer-readable recording medium stores a program code. The program code is loaded onto a processor to perform the aforementioned steps.

Based on the above, the clinical recommendation method, a clinical recommendation apparatus, and a computer-readable recording medium according to the embodiments of the disclosure may obtain medical parameters which are independent events and provide a recommendation according to the probabilities of multiple diagnoses. Therefore, multiple clinical-related data would be considered, so as to provide a proper recommendation.

To make the aforementioned more comprehensible, several embodiments accompanied with drawings are described in detail as follows.

### BRIEF DESCRIPTION OF THE DRAWINGS

The accompanying drawings are included to provide a further understanding of the disclosure, and are incorporated in and constitute a part of this specification. The drawings illustrate exemplary embodiments of the disclosure and, together with the description, serve to explain the principles of the disclosure.

FIG. 1 is a block diagram of elements of a clinical recommendation apparatus according to an embodiment of the disclosure.

FIG. 2 is a flow chart of a clinical recommendation method according to an embodiment of the disclosure.

### DESCRIPTION OF THE EMBODIMENTS

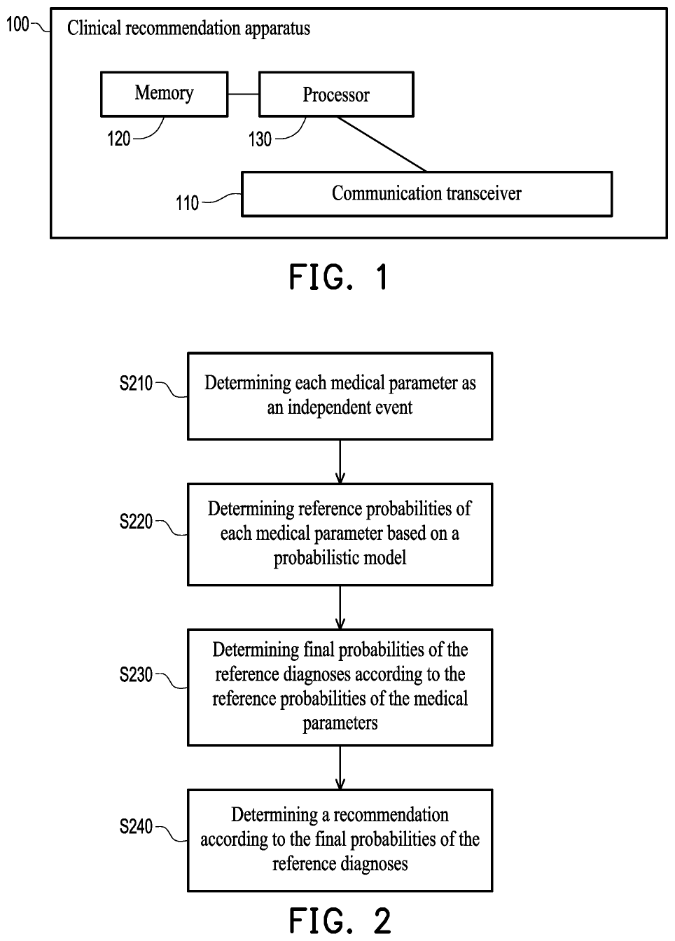

FIG. 1 is a block diagram of elements of a clinical recommendation apparatus 100 according to one of the exemplary embodiments of the disclosure. Referring to FIG. 1, the clinical recommendation apparatus 100 includes (but is not limited to) a communication transceiver 110, a memory 120, and a processor 130. The clinical recommendation apparatus 100 may be a desktop computer, a laptop, a smartphone, a tablet computer, a wearable device, a server, a medical testing instrument, a smart assistance device, or other computing devices.

The communication transceiver 110 may be a transceiver supporting fifth-generation (5G) or another-generation mobile communication, Wi-Fi, Bluetooth, infrared, radio frequency identification (RFID), Ethernet, optical fiber network, etc. Alternatively, the communication transceiver 110 may be a transmission interface such as universal serial bus (USB), thunderbolt, or other communication transmission interfaces. In an embodiment of the disclosure, the communication transceiver 110 is configured to transmit data to or receive data from other electronic devices (for example, a data server, a database, or a storage carrier).

The memory 120 may be any type of fixed or removable random access memory (RAM), read-only memory (ROM), flash memory, hard disk drive (HDD), solid-state drive (SSD), or a similar element. In an embodiment, the memory 120 is used to store program codes, software modules, configurations, data (for example, medical parameters, probabilities, association coefficients, models, etc.), or files, and the embodiment will be described in detail later.

The processor 130 is coupled to the communication transceiver 110 and the memory 120. The processor 130 may be a central processing unit (CPU), a graphics processing unit (GPU), or other programmable general-purpose or special-purpose elements such as a microprocessor, a digital signal processor (DSP), a programmable controller, a field programmable gate array (FPGA), an application-specific integrated circuit (ASIC), a neural network accelerator or other similar elements or a combination of the above elements. In an embodiment, the processor 130 is used to execute all or part of the operations of the clinical recommendation apparatus 100 and may load and execute various program codes, software modules, files, and data stored by the memory 120. The functions of the processor 130 may also be implemented by an independent electronic device or an integrated circuit (IC), and operations of the processor 130 may also be implemented by software.

The elements, units, and modules in clinical recommendation apparatus 100 are applied in the following embodiments to explain the method related to medication list management provided herein. Each step of the method can be adjusted according to actual implementation situations and should not be limited to what is described herein.

FIG. 2 is a flow chart of a clinical recommendation method according to one of the exemplary embodiments of the disclosure. Referring to FIG. 2, the processor 130 determines each of multiple medical parameters as an independent event (step S210). Specifically, the medical parameters may include at least one of a medication record, a surgery record, a treatment record, an examination report, a consultant record, an emergency service record, a disease record, a discharge medical record, and an admission medical record. For example, the medication record includes medication, dosage, and/or schedule of taking medicine. The examination report includes text, numbers, images, or signal waveform. The surgery/treatment record includes surgery/treatment type and/or tool/device. The consultant record includes symptoms and/or problems. However, the content of the medical parameter is not limited thereto in the embodiments of the disclosure. The medical parameter may be obtained from another device such as a database or a server through the communication transceiver 110 or from medical/examination equipment.

In one embodiment, the processor 130 may obtain a key vocabulary from a clinical record of the medication record, the surgery record, the treatment record, the examination report, the consultant record, the emergency service record, the disease record, the discharge medical record, or the admission medical record based on natural language processing. The clinical record may be made by oral, handwriting, or typing. The raw data of the clinical record may not be understood by the processor 130. Natural language processing (NLP) may try to find out how computers interact with human language and further process and analyze large amounts of natural language data. In addition, natural language generation (NLG) is a subfield of NLP. NLG attempts to understand input sentences to generate a machine representation language and further convert the representation language into words. For example, an NLP model embeds words into a low-dimensional space and encodes the relationship between words, encodes word vectors into a vector considering context and semantics through techniques such as RNN, and places attention on important words. Therefore, key vocabulary outputted from the NLP model is generated. Then, the processor 130 may determine the key vocabulary as one of the medical parameters. In one embodiment, the NLP model may refer to a vocabulary system such as SNOMED, UMNLS, or ICD to generate the key vocabulary. For example, the key vocabulary may be one of the clinical terms defined by the vocabulary system. However, in some embodiments, the key vocabulary is the original vocabulary segmented from the sentence recorded in the clinical record.

In one embodiment, the examination report may include one or more numeric type variables. “Positive” is defined as normal, and “Negative” is defined as abnormal. Variables would be labeled as “lab order”, “Positive”, “Negative”, “Positive High”, “Positive Low”, “Positive Extreme High”, or “Positive Extreme Low” with extreme upper and lower limits. Each variable with a label would be considered as one medical parameter.

In one embodiment, when or only when one or more medicines or examinations are taken in multiple days, medicine or examination on each day would be considered as one medical parameter, respectively. For example, the medicine taken on the first day, the second day, and the third day would be considered as three medical parameters.

Furthermore, each medical parameter has an actual diagnosis. The actual diagnosis may be, for example, a disease, a lesion, a physiological status, or a psychological status. The actual diagnosis is the diagnosis confirmed by a doctor, an examiner, or nursing personnel. For example, the actual diagnosis is obtained from a medical record or a clinical record.

On the other hand, the independent event is defined that one of the medical parameters is independent of its actual diagnosis. It should be noticed that in the theory of conditional probability, if the probability of a first event under the condition that a second event occurs equals the (unconditional) probability of the first event, the first and second events would be considered independent. That is, the first event is independent of the second event. In one embodiment, each medical parameter is independent of its actual diagnosis.

In one embodiment, the processor 130 may determine the independent event according to an association coefficient between each of the medical parameters and its actual diagnosis, where the association coefficient is less than a threshold. The association coefficient may be determined through an evaluation model trained through a machine learning algorithm. The machine learning algorithm may be a supervised learning algorithm or an unsupervised learning algorithm. The machine learning algorithm may analyze training samples to obtain features from the training samples, so as to predict unknown data based on the features. The evaluation model is a machine learning model constructed after being trained, and inferences on the data to be evaluated are made based on the evaluation model.

In one embodiment, the evaluation model uses actual diagnoses and medical parameters as the training samples. In addition, the association coefficients are related to the degree of association between actual diagnoses and medical parameters. For example, a higher association coefficient indicates a higher degree of association between a medical parameter and a diagnosis, and may mean it would be a higher chance that the diagnosis is made in the condition of the medical parameter (but not limited to thereto). Alternatively, a lower association coefficient indicates a lower degree of association between a medical parameter and a diagnosis, and may mean it would be a lower chance that the diagnosis is made in the condition of the medical parameter (but not limited to thereto).

For example, in Document 1, “Improved diagnosis medicine association mining to reduce pseudo-associations” accepted in Computer Methods and Programs in Biomedicine in May 2021, a secondary coefficient is used to calculate the association coefficients between diagnosis and medicines and the association coefficients between diagnosis and tests, thereby reducing false associations.

In one embodiment, the evaluation model is a probabilistic model. The probabilistic model is an unsupervised learning algorithm and is an important method of data mining. For example, in Document 2, “An automated technique for identifying associations between medications, laboratory results and problems” on pages 891 to 901 of Biomedical Information Journal, volume 43, issue 6, December 2020, a conviction coefficient is used to calculate the association coefficients between diagnosis and medicines and the association coefficients between diagnosis and tests, and the relationship between the above two pairs of clinical variables (for example, diagnosis and medicines, and diagnosis and tests) may be determined based on the strength of the association coefficients.

As another example, in Document 3, “A Probabilistic Model for Reducing Medicine Errors” published by https://doi.org/10.1371/journal.pone.0082401 in December 2013, a similar model Q coefficient is used to calculate the correlation coefficients between diagnosis and medicines and the correlation coefficients between medicines and medicines, and an appropriateness of prescription (AOP) model is used to evaluate the appropriateness of a prescription. A range of the Q coefficient is defined within [0,∞]; Q=1 means that there is no association between a disease and a medicine; Q<1 means that a disease and medicine are negatively correlated; and Q>1 means that a disease and medicine are positively correlated.

In another embodiment, the evaluation model is a neural network model. For example, a deep neural network (DNN). This deep neural network architecture includes an input layer, a hidden layer, and an output layer. It is to be noted that the deep neural network is formed by a multi-layer neuron structure, and each layer of neurons is configured with an input (for example, an output of a previous layer of neurons) and an output. The neurons in any layer of the hidden layer, through the inner product of an input vector and a weight vector, output a scalar result through a nonlinear transfer function. In the learning stage of the evaluation model, the aforementioned weight vector is trained and determined. Alternatively, while in the inference stage of the evaluation model, the determined weight vector is used to obtain an evaluation result (that is, the output). In this embodiment, the evaluation result of the evaluation model is the association coefficient between input variables. The association coefficient may be a probability, Q coefficient, or other quantized values. The input variables include, for example, a medical parameter and a diagnosis.

In one embodiment, the processor 130 may maintain the association between a medical parameter and its actual diagnosis having the highest association coefficient from multiple association coefficients and disconnect other associations which do not have the highest association coefficient. Then, modified association coefficients between multiple medical parameters and multiple diagnoses would be generated through the evaluation model based on the aforementioned maintaining and disconnecting manners.

In response to the association coefficients being determined, the processor 130 may further exclude the medical parameter having an association coefficient larger than a threshold. For example, general-purpose pain relievers and fever reducers such as Acetaminophen or Aspirin may be related to multiple diseases or symptoms, and their association coefficients would be larger than the threshold. Based on the discrete condition of the association coefficients, those medication records (i.e., the medical parameters) including the general-purpose pain reliever and fever reducer would be excluded.

Eventually, the remaining medical parameters considered as the independent event would have association coefficients less than or equal to the threshold. The excluded medical parameters would not be the independent event and be ignored from the subsequent recommendation determination. The threshold could be 1, 1.5, or another number depending on the type of evaluation module or coefficient-related algorithm.

The processor 130 determines multiple reference probabilities of each of the medical parameters based on a probabilistic model (step S220). Specifically, the probabilistic model could be the aforementioned model of document 2, document 3, a model in conjunction with the aforementioned maintaining and disconnecting manners, or another probabilistic model related to medical data. Each reference probability is a probability of one of the medical parameters in a condition where one of the reference diagnoses occurs. The reference diagnoses are the predefined diagnoses. For example, specific diseases, specific physiological statuses, or specific psychological statuses.

The processor 130 determines multiple final probabilities of the reference diagnoses according to the reference probabilities of the medical parameters (step S230). Specifically, each final probability is a probability of one reference diagnosis under the condition that those medical parameters occur. For example, the reference diagnoses include Type 2 diabetes mellitus and Hypertension. Therefore, two final probabilities corresponding to two reference diagnoses under the condition that multiple medical parameters occur are generated.

In one embodiment, the processor 130 may determine an unconditional probability of each of the reference diagnoses, an unconditional probability of each of the medical parameters, and a joint probability of each of the reference diagnoses with each of the reference diagnoses. The unconditional probability is the probability of an event without considering other events. The joint probability is the probability of multiple events being true or multiple events occurring. The processor 130 may determine the final probabilities of the reference diagnoses according to the unconditional probability of each of the reference diagnoses, the unconditional probability of each of the medical parameters, and the joint probability of each of the reference diagnoses with each of the reference diagnoses. The characteristic of the independent event is that the probability of a first event under the condition that a second event occurs equals the (unconditional) probability of the first event. Therefore, the final probability would be related to a multiplication of the unconditional probabilities of the medical parameters.

In one embodiment, assuming there are i medical parameters, the final probability is determined as follows.

$$P ( D \vert {M}_{1} , {M}_{2} , … , {M}_{i} ) = \frac{P ( {M}_{1} , {M}_{2} , … , {M}_{i} , D )}{P ( {M}_{1} , {M}_{2} , … , {M}_{i} )} = \frac{P ( {M}_{1} , {M}_{2} , … , {M}_{i} , D )}{P ( {M}_{1} ) P ( {M}_{2} ) … P ( {M}_{i} )} \qquad ( 1 )$$

$$P ( {M}_{1} , {M}_{2} , … , {M}_{i} , D ) = P ( {M}_{1} \vert D , {M}_{2} , … , {M}_{i} ) P ( D , {M}_{2} , … , {M}_{i} ) = P ( {M}_{1} \vert D ) P ( D , {M}_{2} , … , {M}_{i} ) = P ( {M}_{1} \vert D ) P ( {M}_{2} \vert D ) … P ( {M}_{i - 1} \vert D ) P ( D , {M}_{i} ) \qquad ( 2 )$$

$$P ( D \vert {M}_{1} , {M}_{2} , … , {M}_{i} ) = \frac{P ( {M}_{1} \vert D ) P ( {M}_{2} \vert D ) … P ( {M}_{i - 1} \vert D ) P ( D , {M}_{i} )}{P ( {M}_{1} ) P ( {M}_{2} ) … P ( {M}_{i} )} = P ( D ) {Q}_{DM 1} {Q}_{DM 2} … {Q}_{DMi} \\ P ( D \vert {M}_{1} , {M}_{2} , … , {M}_{i} ) is a final probability of one \\ of the reference diagnoses , P ( {M}_{1} , {M}_{2} , … , {M}_{i} , D ) \qquad ( 3 )$$

is the joint probability of the reference diagnosis with the i medical parameters, D is the reference diagnosis, Mᵢ is i-th medical parameter, i is an integer, P(D) is the unconditional probability of the reference diagnosis, Q_{DMi} is

$$\frac{P ( {M}_{i} , D )}{P ( {M}_{i} ) P ( D )} ,$$

P(Mᵢ) is the unconditional probability of the i-th medical parameter, and P(Mᵢ,D) is the joint probability of the reference diagnosis with the i-th medical parameter.

The processor 130 determines a recommendation according to the final probabilities of the reference diagnoses (step S240). Specifically, the recommendation could be a list of reference diagnoses with their final probabilities, an order of the final probabilities with their reference diagnoses, or one or more reference diagnoses having the highest final probability. Assuming multiple medical parameters of one patient are considered, the recommendation for this patient would be generated.

For example, table (1) are medical parameters and their reference probabilities/association coefficients.

TABLE 1

| Medical parameter | PCO2 | PH | PO2 | BaseExcess |
|---|---|---|---|---|
| Reference probability | 7.509 | 6.678 | 5.146 | 4.522 |

| | Medical parameter | | | |
|---|---|---|---|---|
| | | | Piperacillin | |
| | | | and beta- | |
| | Insulin | | lactamase | |
| | aspart | Terbutaline | inhibitor | Dexamethasone |
| Reference | 3.952 | 3.744 | 3.638 | 3.358 |
| probability | | | | |

The final probability of acute respiratory failure in hypercapnia could be 0.001796172*7.509*6.678*5.146*4.522*4.139*3.952*3.744*3.638*3.358=1568.08056. The final probability of ventilator-associated pneumonia could be 0.00079063344927398*7.509*6.678*5.146*4.522*4.139*3.952*3.744*3.638*3.358=690.232862. However, in a conventional way such as weighted integration of associations, diagnoses such as acidosis, respiratory failure, pneumonia, hyperkalemia, diabetes, asthma, pneumococcal Streptococcus infection, and arthritis would be recommended.

In addition, the disclosure further provides a non-transitory computer-readable recording medium (e.g., a storage medium such as a hard disk, a compact disk, a flash memory, or a solid state disk (SSD)). The computer-readable recording medium is capable of storing a plurality of code segments (e.g., code segments of storage space detection, code segments of spatial adjustment option presentation, code segments of maintenance work, and code segments of frame presentation, etc.). After the code segments are loaded onto the processor 130 or another processor and executed, all the steps of the above clinical recommendation method can be completed.

In summary, in the clinical recommendation method, the clinical recommendation apparatus, and the non-transitory computer-readable recording medium of the embodiment of the disclosure, the medical parameters of independent events are obtained, and the recommendation is provided based on structuralized and unstructuralized events. Accordingly, missing clinical results could be predicted, and the accuracy of prediction could be improved.

It will be apparent to those skilled in the art that various modifications and variations can be made to the disclosed embodiments without departing from the scope or spirit of the disclosure. In view of the foregoing, it is intended that the disclosure covers modifications and variations provided that they fall within the scope of the following claims and their equivalents.

1. A clinical recommendation method, implemented by a processor, and the clinical recommendation method comprising:
determining each of a plurality of medical parameters as an independent event, wherein each of the plurality of medical parameters has an actual diagnosis, and the independent event is defined that one of the plurality of medical parameters is independent of the actual diagnosis thereof;
determining a plurality of reference probabilities of each of the plurality of medical parameters based on a probabilistic model, wherein each of the plurality of reference probabilities is a probability of one of the plurality of medical parameters in a condition that one of the plurality of reference diagnoses occurs;
determining a plurality of final probabilities of the plurality of reference diagnoses according to the plurality of reference probabilities of the plurality of medical parameters, comprising:
determining an unconditional probability of each of the plurality of reference diagnoses, an unconditional probability of each of the plurality of medical parameters, and a joint probability of each of the plurality of reference diagnoses with each of the plurality of reference diagnoses; and
determining the plurality of final probabilities of the plurality of reference diagnoses according to the unconditional probability of each of the plurality of reference diagnoses, the unconditional probability of each of the plurality of medical parameters, and the joint probability of each of the plurality of reference diagnoses with each of the plurality of medical parameters; and
determining a recommendation according to the plurality of final probabilities of the plurality of reference diagnoses.

2. (canceled)

3. The clinical recommendation method according to claim 1, wherein
$$P ( D \vert {M}_{1} , {M}_{2} , … , {M}_{i} ) = P ( D ) {Q}_{DM 1} {Q}_{DM 2} … {Q}_{DMi} ,$$
P(D|M₁, M₂, . . . , Mᵢ) is a final probability of one of the plurality of reference diagnoses, D is the one of the plurality of reference diagnoses, Mᵢ is i-th medical parameter, i is an integer, P(D) is the unconditional probability of the one of the plurality of reference diagnoses, Q_{DMi} is
$$\frac{P ( {M}_{i} , D )}{P ( {M}_{i} ) P ( D )} ,$$
P(Mᵢ) is the unconditional probability of the i-th medical parameter, and P(Mᵢ,D) is the joint probability of the one of the plurality of reference diagnoses with the i-th medical parameter.

4. The clinical recommendation method according to claim 1, wherein the plurality of medical parameters comprise at least one of a medication record, a surgery record, a treatment record, an examination report, a consultant record, an emergency service record, a disease record, a discharge medical record, and an admission medical record.

5. The clinical recommendation method according to claim 4, further comprising:
obtaining a key vocabulary from a clinical record of the medication record, the surgery record, the treatment record, the examination report, the consultant record, the emergency service record, the disease record, the discharge medical record, or the admission medical record based on natural language processing; and
determining the key vocabulary as one of the plurality of medical parameters.

6. The clinical recommendation method according to claim 1, wherein determining each of the plurality of medical parameters as the independent event comprises:
determining the independent event according to an association coefficient between each of the plurality of medical parameters and the actual diagnosis thereof, wherein the association coefficient is less than a threshold; and
excluding another medical parameter having an association coefficient larger than the threshold.

7. A clinical recommendation apparatus, comprising:
a memory, configured to store a program code; and
a processor, coupled to the memory, and configured to execute the program code to perform:
determining each of a plurality of medical parameters as an independent event, wherein each of the plurality of medical parameters has an actual diagnosis, and the independent event is defined that one of the plurality of medical parameters is independent of the actual diagnosis thereof;
determining a plurality of reference probabilities of each of the plurality of medical parameters based on a probabilistic model, wherein each of the plurality of reference probabilities is a probability of one of the plurality of medical parameters in a condition that one of the plurality of reference diagnoses occurs;
determining a plurality of final probabilities of the plurality of reference diagnoses according to the plurality of reference probabilities of the plurality of medical parameters, comprising:
determining an unconditional probability of each of the plurality of reference diagnoses, an unconditional probability of each of the plurality of medical parameters, and a joint probability of each of the plurality of reference diagnoses with each of the plurality of reference diagnoses; and
determining the plurality of final probabilities of the plurality of reference diagnoses according to the unconditional probability of each of the plurality of reference diagnoses, the unconditional probability of each of the plurality of medical parameters, and the joint probability of each of the plurality of reference diagnoses with each of the plurality of medical parameters; and
determining a recommendation according to the plurality of final probabilities of the plurality of reference diagnoses.

8. (canceled)

9. The clinical recommendation apparatus according to claim 7, wherein
$$P ( D \vert {M}_{1} , {M}_{2} , … , {M}_{i} ) = P ( D ) {Q}_{DM 1} {Q}_{DM 2} … {Q}_{DMi} ,$$
P(D|M₁, M₂, . . . , Mᵢ) is a final probability of one of the plurality of reference diagnoses, D is the one of the plurality of reference diagnoses, Mᵢ is i-th medical parameter, i is an integer, P(D) is the unconditional probability of the one of the plurality of reference diagnoses, Q_{DMi} is
$$\frac{P ( {M}_{i} , D )}{P ( {M}_{i} ) P ( D )} ,$$
P(Mᵢ) is the unconditional probability of the i-th medical parameter, and P(Mᵢ,D) is the joint probability of the one of the plurality of reference diagnoses with the i-th medical parameter.

10. The clinical recommendation apparatus according to claim 7, wherein the plurality of medical parameters comprise at least one of a medication record, a surgery record, a treatment record, an examination report, a consultant record, an emergency service record, a disease record, a discharge medical record, and an admission medical record.

11. The clinical recommendation apparatus according to claim 10, wherein the processor further performs:
obtaining a key vocabulary from a clinical record of the medication record, the surgery record, the treatment record, the examination report, the consultant record, the emergency service record, the disease record, the discharge medical record, or the admission medical record based on natural language processing; and
determining the key vocabulary as one of the plurality of medical parameters.

12. The clinical recommendation apparatus according to claim 7, wherein the processor further performs:
determining the independent event according to an association coefficient between each of the plurality of medical parameters and the actual diagnosis thereof, wherein the association coefficient is less than a threshold; and
excluding another medical parameter having an association coefficient larger than the threshold.

13. A non-transitory computer-readable recording medium, storing a program code, the program code being loaded onto a processor to perform:
determining each of a plurality of medical parameters as an independent event, wherein each of the plurality of medical parameters has an actual diagnosis, and the independent event is defined that one of a plurality of medical parameters is independent of the actual diagnosis thereof;
determining a plurality of reference probabilities of each of the plurality of medical parameters based on a probabilistic model, wherein each of the plurality of reference probabilities is a probability of one of the plurality of medical parameters in a condition that one of the plurality of reference diagnoses occurs;
determining a plurality of final probabilities of the plurality of reference diagnoses according to the plurality of reference probabilities of the plurality of medical parameters, comprising:
determining an unconditional probability of each of the plurality of reference diagnoses, an unconditional probability of each of the plurality of medical parameters, and a joint probability of each of the plurality of reference diagnoses with each of the plurality of reference diagnoses; and
determining the plurality of final probabilities of the plurality of reference diagnoses according to the unconditional probability of each of the plurality of reference diagnoses, the unconditional probability of each of the plurality of medical parameters, and the joint probability of each of the plurality of reference diagnoses with each of the plurality of medical parameters; and
determining a recommendation according to the plurality of final probabilities of the plurality of reference diagnoses.
